SEQUENCE LISTING (14 sequences)

SEQ ID NO: 1 - protein, 21 aa, Artificial Sequence
  Synthetic
CSCSSLMDKECVYFCHLDIIW

SEQ ID NO: 2 - protein, 14 aa, Artificial Sequence
  Synthetic
DEEAVYFAHLDIIW

SEQ ID NO: 3 - protein, 18 aa, Artificial Sequence
  Synthetic
ACLMDKEAVYFAHLDIIW

SEQ ID NO: 4 - protein, 21 aa, Artificial Sequence
  Synthetic
CSCKDMTDKECLNFCHQDVIW

SEQ ID NO: 5 - protein, 21 aa, Artificial Sequence
  Synthetic
CSCKDMTDKECLYFCHQDVIW

SEQ ID NO: 6 - protein, 21 aa, Artificial Sequence
  Synthetic
CTCNDMTDEECLNFCHQDVIW

SEQ ID NO: 7 - protein, 21 aa, Artificial Sequence
  Synthetic
CTCNDMTDKECLYFCHQDIIW

SEQ ID NO: 8 - protein, 5 aa, Artificial Sequence
  Synthetic
DPVLW

SEQ ID NO: 9 - protein, 6 aa, Artificial Sequence
  Synthetic
LWAAYF

SEQ ID NO: 10 - protein, 5 aa, Artificial Sequence
  Synthetic
DPILW

SEQ ID NO: 11 - protein, 5 aa, Artificial Sequence
  Synthetic
SPVLW

SEQ ID NO: 12 - protein, 5 aa, Artificial Sequence
  Synthetic
EAVLW

SEQ ID NO: 13 - protein, 5 aa, Artificial Sequence
  Synthetic
EAILW

SEQ ID NO: 14 - protein, 21 aa, Artificial Sequence
  Synthetic
ALCNCNRIIIPHMCWKKCGKK

(19) United States
(12) Patent Application Publication
(10) Pub. No.: US 2003/0104976 A1
(43) Pub. Date: Jun. 5, 2003

(54) Analgesic methods using endothelin receptor ligands

(76) Inventors: Gudarz Davar, Jamaica Plain, MA (US); Gary Strichartz, Sherborn, MA (US); Moin Fareed, Randolph, MA (US); Alla Khodorova, Brighton, MA (US)

Correspondence Address: CLARK & ELBING LLP, 101 FEDERAL STREET, BOSTON, MA, 02110, US

(21) Appl. No.: 10/200,923

(22) Filed: Jul. 23, 2002

Related U.S. Application Data
(60) Provisional application No. 60/307,228, filed on Jul. 23, 2001.

(51) Int. Cl. A61K 31/00; A61K 31/44

(52) U.S. Cl. 514/1

(57) ABSTRACT

The present invention features methods and compositions for preventing, reducing or eliminating pain (for example, acute pain) using an endothelin-B receptor agonist alone or in combination with either an endothelin-A receptor antagonist, an opioid receptor agonist, a GIRK channel activator, or a PKC activator.

### CROSS REFERENCE TO RELATED APPLICATIONS

This application claims benefit of the filing date of the copending U.S. Provisional Application No. 60/307,228 (filed Jul. 23, 2001), hereby incorporated by reference.

This invention was made with the support of the United States government under USPHS Grant No. CA 80153. The United States may have certain rights in the invention.

### FIELD OF THE INVENTION

This invention features methods and pharmaceutical compositions for treating pain.

### BACKGROUND OF THE INVENTION

Nociception, or the sensation of pain, is a common symptom indicative of an underlying disease or injury and is often the primary symptom for which treatment is sought. Pain can take a variety of forms depending on the underlying cause or the type of painful stimulus. Acute pain is generally classified as a temporary pain that is caused by tissue damage, most commonly associated with trauma or surgery. Normally, acute pain disappears as a damaged tissue heals and can typically last anywhere from a few seconds to many months. Chronic pain can persist even after a tissue has healed, and lasts from a few weeks to many years. Chronic pain is also associated with chronic disease states like cancer.

Endothelin-1 (ET-1) is an endogenous, 21 amino acid peptide that is a potent vasoconstrictor. In vascular tissues, the actions of ET-1 are mediated by two distinct G-protein-coupled receptors; the endothelin-A (ET_{A}) and the endothelin-B (ET_{B}) receptor which usually affect vasoconstriction and vasodilation, respectively. Of the two receptor subtypes, the ET_{A} receptor has the higher affinity for ET-1.

Although most efforts to establish a function for ET-1 have focused on its potential importance in cardiovascular disease, there is support for a role for ET-1 in the pathogenesis of pain. Clinical findings suggest that painful sensations are evoked by ET-1 dysregulation in a variety of conditions. In human patients, elevated plasma ET-1 levels are correlated with pain severity in several malignant neoplastic and painful ischemic conditions (Nelson et al., Nature Medicine, 1: 944-949, 1995; Graido-Gonzalez et al., Blood 92: 2551-2555, 1998). Metastatic prostate and breast cancer cells secrete high concentrations of ET-1 and the painful sensation can be blocked by systemic administration of an ET_{A} receptor antagonist (Kopetz et al., Invest. New Drugs 20: 173-182, 2002). Exogenous ET-1 can also produce pain in human subjects. ET-1 injection into the brachial artery induces severe pain and prolonged, touch-evoked allodynia (Dahlof et al., J. Hypertension 8: 811-818, 1990).

### SUMMARY OF THE INVENTION

The present invention provides methods and compositions for treating (i.e., preventing, reducing, or eliminating) pain in a mammal (for example, a human) by administering an analgesia-inducing amount of an endothelin-B receptor (ET_{B}) agonist. The painful conditions treated by the methods and compositions of the present invention are induced by elevated ET-1 levels either systemically or locally and can result, for example, from myocardial infarction, angina, ischemic cardiovascular disease, sickle cell anemia, migraine headache, peripheral vascular occlusive disease, metastatic prostate or breast cancer, inflammatory conditions of the skin or joints, diabetic neuropathy, peripheral arterial occlusive disease, or acute tissue damage from surgery or traumatic injury.

In particularly useful embodiments, the ET_{B} receptor agonist is administered topically to treat conditions of the skin and/or joints. Medical conditions particularly amenable to topical treatment using ET_{B} receptor agonists include cutaneous damage such as that resulting from traumatic injury or surgery, chemical, thermal, or radiation burns, including sunburn, lesions to the dermis, epidermis, or underlying tissue, and painful cutaneous conditions including, for example, psoriasis, scleroderma, or pruritis. Typically, the ET_{B} receptor agonist is formulated as a cream, spray, or ointment. Alternatively, bandages, gauze, or other wound dressings can be impregnated (e.g., soaked) in a solution containing the ET_{B} receptor agonist prior to application to the affected site.

A particularly useful endothelin-B receptor agonist is IRL-1620. Other suitable ET_{B} receptor agonists include, for example, BQ-3020, sarafotoxin S6a, sarafotoxin S6b, sarafotoxin S6c, and sarafotoxin S6d.

Optionally, the pain can be treated by combining an endothelin-B receptor agonist with other analgesia-inducing compounds. Suitable analgesia-inducing compounds that can be used in combination with an endothelin-B receptor agonist include endothelin-A receptor antagonists, opioid receptor agonists, GIRK channel activators, and PKC activators. Particularly useful endothelin-A receptor antagonists include, for example, sulfisoxazole and ABT-627 (atrasentan; 2R-(4-methoxyphenyl)-4S-(1,3-benzodioxol-5-yl)-1-(N, N-di(n-butyl)aminocarbonyl-methyl)-pyrrolidine-3R-carboxylic acid). Other suitable ET_{A} receptor antagonists include, for example, BQ-123, BQ-610, SB 209670, SB 217242, FR-139317, PD-151242, TTA-386, JKC-301, JKC-302, BE-18257A, BE-18257B, BQ-485, TBC-11251, PD 156707, A-127722, and LU 135252. Particularly useful opioid agonists include, for example, morphine, codeine, hydrocodone, and oxycodone.

When a second analgesia-inducing compound is administered in combination with an endothelin-B receptor agonist, according to the methods of this invention, it is preferable that the two compounds are administered within 24 hours, 12 hours, or 1 hour of each other, or simultaneously. Alternatively, the two compounds may be administered in the same pharmaceutical formulation.

Any effective route of administration for the endothelin-B receptor agonist and the optional second analgesia-inducing compound may be used. Preferred routes of administration include intravenous, intramuscular, and subcutaneous injection, as well as oral and topical administration. In cases where a second analgesia-inducing compound is administered in combination with an endothelin-B receptor agonist for treating pain, the two compounds need not be administered by the same route.

By “ET_{B} receptor agonist” is meant any naturally occurring or synthetic compound that binds to the ET_{B} receptor and mimics the function of ET-1 at that receptor. To be considered an ET_{B} receptor agonist, partial efficacy is sufficient (i.e., partial agonists). The ET_{B} receptor agonist may be a peptide or a non-peptide compound. Preferably, ET_{B} receptor agonists have a dissociation constant (K_{d}) for the ET_{B} receptor of <1 μM, more preferably <100 nM, most preferably <10 nM, or even <1 nM. ET_{B} receptor agonists include, for example, IRL-1620, BQ-3020, sarafotoxin S6a, sarafotoxin S6b, sarafotoxin S6c, and sarafotoxin S6d (Table 2).

By “ET_{A} receptor antagonist” is meant any naturally occurring or synthetic compound that binds to the ET_{A} receptor and blocks or inhibits the function of ET-1 or other agonist at that receptor. The ET_{A} receptor antagonist may be a peptide or a non-peptide compound. Preferably, ET_{A} receptor antagonists have a K_{d} for the ET_{A} receptor of <1 μM, more preferably <100 nM, most preferably <10 nM, or even <1 nM. ET_{A} receptor antagonists include, for example, sulfisoxazole, TBC-1 1251, BQ-123, BQ-610, BQ-745, PD 156707, PD 151242, TTA-386, JKC-301, JKC-302, BE-18257A, BE-18257B, A-1277722, LU 135252, TAK-044, SB 209670, SB 217242, FR139317, and ABT-627 (Table 2; Cheng et al., Ann. Reports in Medicinal Chemistry, Section II, Ch. 7, Endothelin Inhibitors, pages 61-70, 1997, ed. A. M. Doherty, Academic Press, Inc.).

By “opioid receptor agonist” is meant any naturally occurring, semi-synthetic, or synthetic compound that binds to the mu, kappa, or delta opioid receptor subtypes and mimics the function of opioids at these receptors. The opioid receptor agonist may be a peptide or a non-peptide compound. Preferably, opioid receptor agonists have a K_{d} for at least one opioid receptor subtype of <1 μM, more preferably <100 nM, most preferably <10 nM, or even <1 nM. Opioid receptor agonists include generally, for example, members from the phenanthrene, phenyl heptylamine, phenylpiperidine, morphinan and benzomorphan chemical families. Opioid receptor agonists include, for example, morphine, hydormorphone, oxymorphone, codeine, oxycodone, hydrocodon, dextromethorphan, methadone, meperidine, levorphanol, alfentanil, buprenorphine, and butorphanol.

By “GIRK channel activator” is meant any compound that increases potassium efflux across a G-protein inwardly rectifying potassium (GIRK) channel. The increased potassium efflux may result from a direct activation of the GIRK channel, or may occur indirectly such that the compound binding to a molecule other than the GIRK channel results in the increased efflux across the GIRK channel.

By “protein kinase C activator” is meant any compound that increases the catalytic activity of any protein kinase C (PKC) isoform. The preferred catalytic activity that is enhanced is the kinase activity.

By “treating pain” is meant preventing, reducing, or eliminating the sensation of pain in a subject. To treat pain, according to the methods of this invention, the treatment does not necessarily provide therapy for the underlying pathology that is causing the painful sensation. Treatment of pain can be purely symptomatic.

By “an effective amount” is meant an amount of a compound, alone or in a combination according to the invention, required to prevent, reduce, or eliminate the sensation of pain (nociception). The effective amount of active compound(s) used to practice the present invention for therapeutic treatment of pain varies depending upon the manner of administration, the age, and body weight, of the subject as well as the underlying pathology that is causing the pain. Ultimately, the attending physician or veterinarian will decide the appropriate amount and dosage regimen. Such amount is referred to as an “effective” amount.

### BRIEF DESCRIPTION OF THE DRAWINGS

FIG. 1 is a graph showing the mean number of hindpaw flinches per 5 minute period for the first 75 minutes following a subcutaneous injection of ET-1.

*p<0.05; **p<0.01; ***p<0.001; ****p<0.0001.

FIG. 2A is a graph showing the effect of BQ-788 on the mean number of hindpaw flinches per 5 minute period when administered alone or in combination with ET-1. FIG. 2B is a graph showing the effect of IRL-1620 on the mean number of hindpaw flinches per 5 minute period when administered in combination with ET-1.

*p<0.05; **p<0.01; ***p<0.001; ****p<0.0001.

FIG. 3 is a graph showing the total number of flinches in response to ET-1 (2 nmol) injected simultaneously with PBS, BQ-788 (60 nmol), IRL-1620 (2 nmol), naloxone (55 nmol), IRL-16220 plus naloxone, tertiapin (20 pmol), or IRL-1620 plus tertiapin.

*p<0.05.

FIG. 4 is a graph showing the mean maximal flinch frequency (MFF; flinches per 5 minutes) in response to ET-1 (2 nmol) injected simultaneously with PBS, BQ-788 (60 nmol), IRL-1620 (2 nmol), naloxone (55 nmol), IRL-16220 plus naloxone, tertiapin (20 pmol), or IRL-1620 plus teriapin.

FIG. 5 is a representative series of electrophysiological recordings of C-fiber activity following subcutaneous plantar hindpaw injections of (A) ET-1 alone, (B) ET-1 and IRL-1620, and (C) ET-1, IRL-1620, and naloxone. The arrow in (A) indicates the time of ET-1 injection. The first and second arrows in (B) indicate the time of IRL-1620 and IRL-1620/ET-1 injections, respectively. The first and second arrows in (C) indicate the time of IRL-1620/naloxone and ET-1/IRL-1620/naloxone injections, respectively.

FIG. 6 is a series of graphs demonstrating the opioid receptor subtypes responsible for mediating ET_{B} receptor-induced analgesia. FIG. 6A is a graph showing the effect of the μ-opioid receptor subtype specific antagonist, CTOP, on the mean number of ET-1 -induced hindpaw flinches per 5 minute period in the presence and absence of the ET_{B} receptor agonist, IRL-1620. FIG. 6B is a graph showing the total number of hindpaw flinches measured during the 75 minute observation period described in FIG. 6A. FIG. 6C is a graph showing the effect of pretreatment with anti-β-endorphin antiserum on the mean number of ET-1-induced hindpaw flinches per 5 minute period in the presence and absence of IRL-1620.

FIG. 7 is a graph showing the level of extracellular β-endorphin released from cultured primary human keratinocytes treated with IRL-1620.

FIG. 8 is a graph showing the mean duration of hindpaw biting or licking per 5 minute period in wild-type mice and mice lacking a functional GIRK2 gene.

*p<0.03, **p<0.01, ***p<0.002.

FIG. 9 shows the chemical structures of selected ET_{A} receptor antagonists.

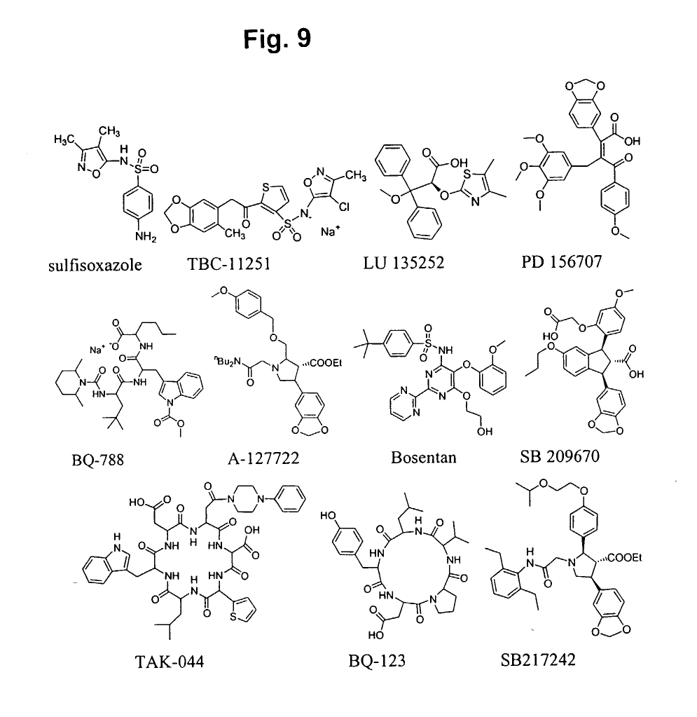

### DETAILED DESCRIPTION

The present invention relates to our discovery that ET_{B} receptor agonists can significantly reduce nociception in a model of acute, ET-1-induced pain and can be used to treat painful conditions associated with abnormally high levels of ET-1. Locally or systemically elevated ET-1 levels can result, for example, from myocardial infarction, angina, ischemic cardiovascular disease, sickle cell anemia, migraine headache, peripheral vascular occlusive disease, metastatic prostate or breast cancer, inflammatory conditions of the skin or joints, diabetic neuropathy, peripheral arterial occlusive disease, or acute tissue damage from surgery or traumatic injury. Pain associated with these conditions are can be treated, reduced, or prevented using the methods and compositions of this invention.

The invention stems from the discovery of the ET_{B} receptor signaling pathway in keratinocytes and its interaction with sensory neurons. We have discovered that stimulation of the keratinocyte ET_{B} receptor results in the increased secretion of the endogenous opioid, β-endorphin, which activates the μ- and κ-opioid receptors located on the nociceptors of neighboring sensory neurons. The neuronal opioid receptors may mediate G-protein coupled inwardly rectifying potassium (GIRK) channel activation, inducing analgesia by causing a membrane hyperpolarization of the nociceptors.

Accordingly, ET_{B} receptor agonists can be used for pain reduction. They may be administered either alone or in combination with other analgesic compounds. For example, we have previously shown that ET_{A} receptor antagonists are useful for treating vasoconstriction-independent ET-1 mediated pain (PCT Publication No. WO 99/56761, hereby incorporated by reference), suggesting that the analgesic effect of ET_{A} receptor antagonists comes from a direct effect on sensory neurons rather than receptors of the microvasculature. Thus, ET_{A} receptor antagonists may be used with ET_{B} receptor agonists for pain reduction.

Alternatively, ET_{B} receptor agonists may be administered in conjunction with opioids. Opioids are among the most powerful clinical analgesics and are frequently prescribed to treat severe pain. Opioid therapy in the clinic, however, often results in undesirable side effects including, nausea, reduced GI motility (constipation), respiratory and CNS depression, and physiological dependence. The present invention, in part, stems from our discovery that naloxone, an opioid receptor antagonist, and an anti-β-endorphin antibody block the analgesic properties of ET_{B} receptor agonists; suggesting that ET_{B} receptor-induced analgesia is mediated by the same or a complementary pathway. Safe and effective analgesic combinations of opioids with ET_{B} receptor agonists can be used to reduce the dosage of opioids required to provide adequate pain relief, thereby minimizing the side effects normally associated with opioid therapy.

We have also discovered that the analgesia-inducing effects of ET_{B} receptor agonists can be inhibited by GIRK channel antagonists. This finding suggests that GIRK channel agonists can also induce analgesia when administered alone, or can augment the effects of other analgesic therapies.

Accordingly, the invention features methods and compositions for treating, reducing, or preventing pain induced by elevated ET-1 levels, using an ET_{B} receptor agonist, either alone or in combination with a second analgesia-inducing agent, such as an ET_{A} receptor antagonist, an opioid receptor agonist, or a GIRK channel activator.

These results are now described in more detail in the following examples. These examples are provided to illustrate the invention and should not be construed as limiting.

### ET_{A} and ET_{B} Receptors Mediate ET-1-induced Pain in the Rat Plantar Hindpaw

An ET1 animal model of acute pain was used to investigate the signaling pathways involved in endothelin receptor-mediated nociception. In this model system, acute pain was induced under sevoflurane anesthesia by subcutaneous ET-1 injection into the rat plantar hindpaw after 40 seconds of limb cooling. Injections were given either as a single 20 μl bolus for single compound or co-administration studies, or as two 10 μl injections separated by 1-2 minutes for pre-treatment studies, and were delivered into the mid plantar paw approximately 1 cm distal to the heel. Repetitive and spontaneous flinching of the ipsilateral hindpaw were counted in 5 minute blocks, beginning 5 minutes after ET-1 injection, for 75 minutes. The time of maximal flinch frequency (MFF) was defined as the 5 minute epoch during which the animal exhibited the greatest number of flinching behaviors. Data was reported as the mean±s.e.m.

In these studies, ET1 (4 nmol) injected into the rat plantar paw evoked ipsilateral hindpaw flinching in 100% of the animals (n=12). Flinching began 10-20 minutes after injection and MFF was reached at 40.8±2.8 minutes. The MFF following a 4 nmol ET1 injection was 40.2±3.9 flinches per 5 minutes. The nociceptive effect of ET1 was transient, with flinching behavior returning to baseline by 75 minutes. The total number of flinches was 178.5±29.1 over the entire 75 minute observation period (Table 1).

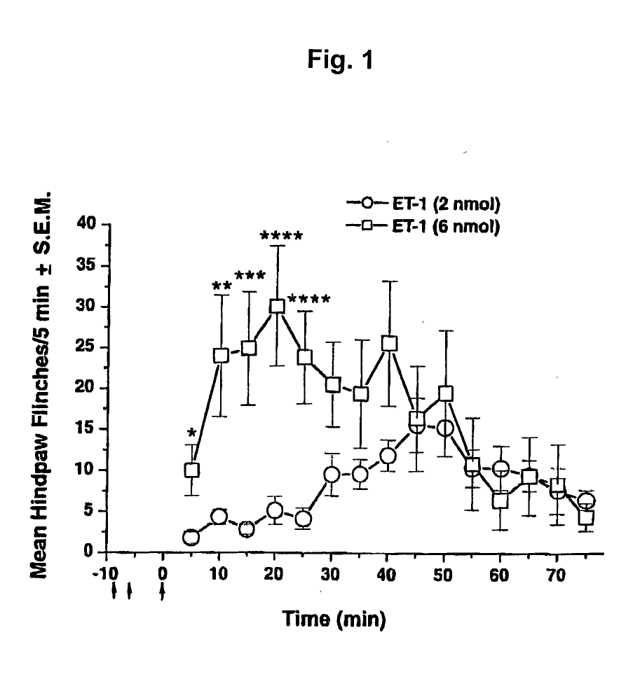

The nociceptive effects of plantar injection of ET1 was dose-dependent (FIG. 1). When effects of 2 nmol ET1 were compared with 6 nmol, the higher dose resulted in a significantly greater amount of flinching (MFF=41.9±8.7 versus 23.8±1.9 flinches per 5 minutes; p<0.05) and a more rapid onset of the MFF (22.5±4.1 versus 51.7±3.1 minutes; p<0.0001; Table 1). The total number of flinches observed over the entire 75 minute observation period was also increased following the higher dose (235.5±66.2 versus 122.8±13.3; p<0.05; Table 1). ET-1 doses of 2 nmol were reliably submaximal and were used for further study.

### ET_{B} Receptor Antagonists

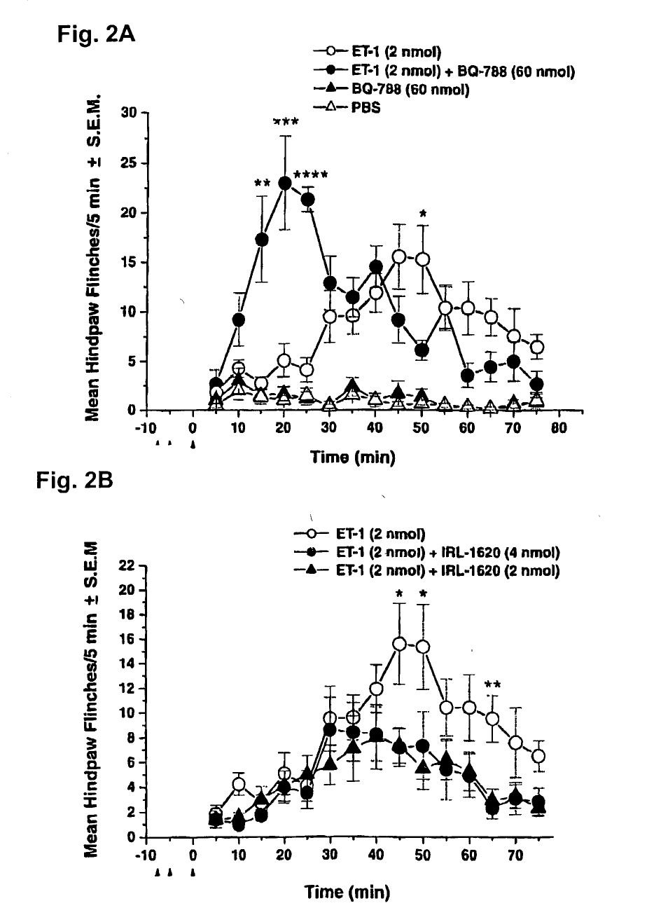

Prior and co-administration of the ET_{B} receptor antagonist, BQ-788 (60 nmol; N-cis-2,6-dimethylpiperidinocarbonyl-L-gamma-methylleucyl-D-1-methoxycarbonyltryptophanyl-D-Nle), with 2 nmol ET-1 accelerated the development of hindpaw flinching. The time to reach MFF was shorter in the presence of BQ-788 (20.0±1.2 versus 51.7±3.1 minutes; p<0.0001; Table 1). BQ-788 also caused a significant increase in flinching frequency at 15, 20, and 25 minute post-injection (FIG. 2A). Neither the duration nor the total number of flinching events was different between the two treatment groups. BQ-788 administered alone either as a single bolus of 60 nmol, or two injections of 30 nmol separated by 1-2 minutes did not result in altered flinching behaviors compared to PBS control.

TABLE 1
Characterization of Endothelin Receptor and Opioid
Receptor Contribution to ET-1-induced Acute Paint†

| | Time to MFF | | Total Number |
|---|---|---|---|
| Treatment‡ | (minutes) | MFF | of Flinches |
| PBS | 25.6 ± 8.9 | 4.0 ± 0.6 | 13.4 ± 1.6 |
| (n = 8) | | | |
| ET-1 | 51.7 ± 3.1 | 23.8 ± 1.9 | 122.8 ± 13.3 |
| (n = 12) | | | |
| ET-1 (4 nmol) | 40.8 ± 2.8 | 40.2 ± 3.9 | 178.5 ± 29.1 |
| (n = 12) | | | |
| ET-1 (6 nmol) | 22.5 ± 4.1 | 41.9 ± 8.7 | 253.3 ± 66.2 |
| (n = 8) | | | |
| BQ-788 | 32.5 ± 10.2 | 5.8 ± 0.9 | 17.3 ± 2.3 |
| (n = 6) | | | |
| ET-1 + BQ-788 | 20.0 ± 1.2 | 30.4 ± 2.2 | 155.6 ± 13.4 |
| (n = 9) | | | |
| ET-1 + IRL-1620 | 37.1 ± 4.0 | 15.7 ± 2.7 | 69.8 ± 15.7 |
| (4 nmol) (n = 12) | | | |
| IRL-1620 (4 nmol) | 33.3 ± 6.1 | 4.7 ± 0.6 | 22.1 ± 3.1 |
| (n = 9) | | | |
| ET-1 + IRL-1620 | 37.5 ± 3.5 | 16.2 ± 2.1 | 66.5 ± 8.5 |
| (n = 12) | | | |
| Naloxone | 28.6 ± 5.6 | 3.3 ± 0.8 | 16.1 ± 4.1 |
| (n = 7) | | | |
| ET-1 + naloxone | 41.3 ± 2.0 | 38.2 ± 4.5 | 146.3 ± 17.1 |
| (n = 12) | | | |
| ET-1 + naloxone + | 40.4 ± 3.3 | 33.7 ± 5.5 | 145.8 ± 19.4 |
| IRL-1620 | | | |
| (n = 12) | | | |
| Tertiapin | N/A | N/A | 17 ± 6 |
| (n = 8) | | | |
| ET-1 + tertiapin | 41 ± 4 | 38 ± 5 | 174 ± 19 |
| (n = 12) | | | |
| ET-1 + tertiapin + | 40 ± 3 | 30 ± 5 | 117 ± 10 |
| IRL-1620 | | | |
| (n = 2) | | | |

†Values are means ± s.e.m.
‡Unless otherwise noted, dosages are: ET-1 = 2 nmol; BQ-788 = 60 nmol; IRL-1620 = 2 nmol; naloxone = 55 nmol; tertiapin = 20 pmol.

### ET_{B} Receptor Agonists

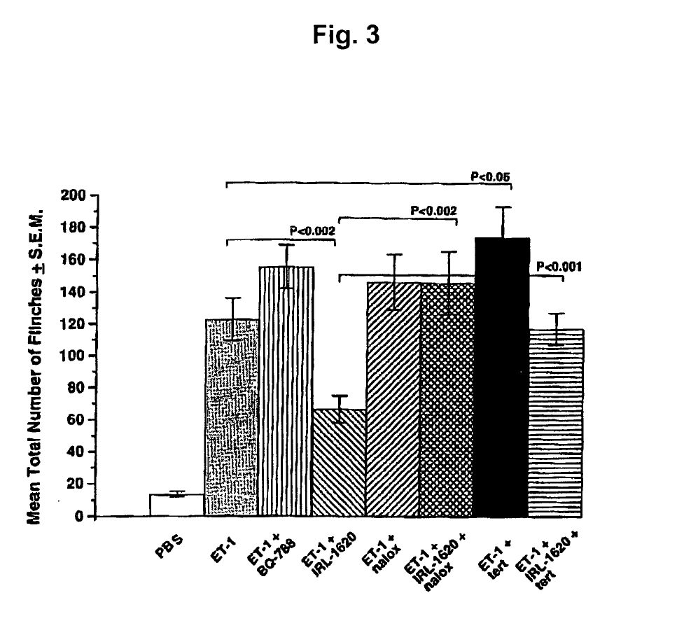

Prior and co-administration of the ET_{B} receptor agonist, IRL-1 620 (4 nmol), with 2 nmol ET1 inhibited the development of hindpaw flinching. The time to reach MFF was shorter in the presence of IRL-1620 (37.1±4.0 versus 51.7±3.1 minutes; p<0.01; Table 1, FIG. 2B). IRL-1620 also caused a significant reduction in the MFF (15.7±2.7 versus 23.8±1.9 flinches per 5 minutes; p<0.05; Table 1, FIG. 2B) and the total number of flinching events (69.8±15.7 versus 122.8±13.3 flinches per 75 minutes; p<0.0²; Table 1, FIG. 3). IRL-1620 (4 nmol) administered alone caused a small but significant increase in flinching behavior compared to PBS control (Table 1). This response is probably attributable to the non-specific actions of IRL-1620 on the ET_{A} receptor at high concentrations. Subsequent experiments were done using 2 nmol IRL-1620 which resulted in the same reduction of ET-1-induced pain behaviors while reducing the experimental variability (Table 1). Thus, ET_{B} receptor activation inhibits pain behavior.

### Modulation of ET_{B} Receptor Signaling by Opioids

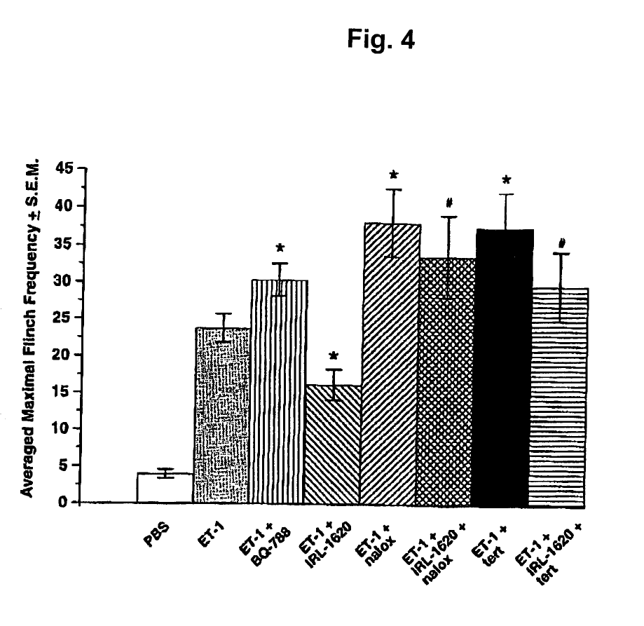

Prior and co-administration of naloxone (55 nmol), a non-selective opioid receptor antagonist, with 2 nmol ET-1 significantly accelerated the development of hindpaw flinching. The time to reach MFF was shorter in the presence of naloxone (41.3±2.0 versus 51.7±3.1 minutes; p<0.01; Table 1). Naloxone also caused a significant increase in the MFF (38.2±4.5 versus 23.8±1.9 flinches per 5 minutes; p<0.01; Table 1, FIG. 4) but not the total number of flinches (Table 1, FIG. 3). Naloxone alone did not induce flinching behavior that was different from PBS control, suggesting that an opioid receptor pathway modulates the ET1-induced pain response.

### Combination of Naloxone and IRL-1620

Prior and co-administration of naloxone (55 nmol) with IRL-1620 (2 nmol) and ET-1 (2 nmol) resulted in a complete reversal of the analgesic effects attributed to IRL-1620 (Table 1, FIGS. 3 and 4). Naloxone administration in combination with ET-1/IRL-1620, prevented the IRL-1620-induced reduction in the total number of flinches (66.5±8.5 versus 145.8±19.4 flinches). The addition of naloxone to the IRL-1620/ET1 combination also prevented the reduction in MFF attributable to IRL-1620 (16.2±2.1 versus 33.7±5.5 flinches per 5 minutes; p<0.01; Table 1). These data indicate that an opioid receptor pathway mediates ET_{B} receptor-induced analgesia because the effects of ET_{B} receptor agonists can be completely blocked by a non-specific opioid antagonist. In addition, these results suggest that the analgesic effects of ET_{B} receptor agonists may be augmented by co-administration of an opioid receptor agonist.

### Electrophysiological Recordings Confirm the ET_{B}-mediated Analgesia

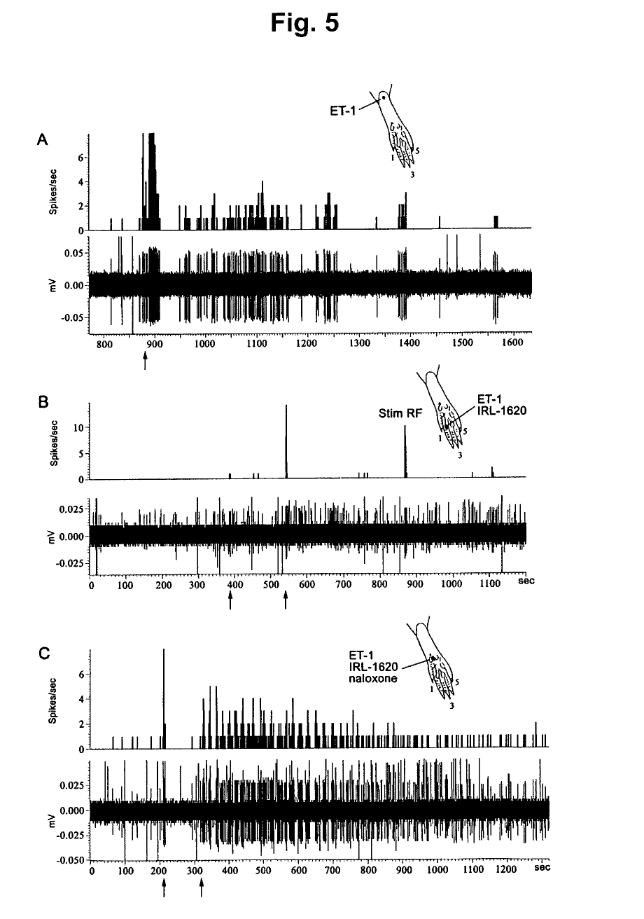

In order to confirm that ET_{B} receptor activation inhibits the ET1-induced pain response and that the inhibition is mediated through an opioid-sensitive mechanism, the spike response of IRL-1620 alone, and in combination with naloxone, were studied in nociceptive C-fibers. Spike activity was recorded in sciatic nerve C-fibers from twelve animals following subcutaneous plantar hindpaw injections of ET-1. Representative recordings of spike activity are provided in FIG. 5. The mean and maximum response frequencies following injection of ET-1 alone (2 nmoles) are 0.32±0.07 imp/s and 4.17±0.17 imp/s, respectively. FIG. 5A shows the characteristic “bursting” pattern of a long-lasting spike discharge. Co-injection of IRL-1620 (2 nmoles) suppressed spike responses to ET1 in all C-fiber units, with mean and maximum response frequencies of 0.08±0.02 imp/s and 1.5±0.4 imp/s, respectively. Noxious pinch stimuli, performed 5.5 minutes after injection, demonstrates continued mechanoresponsiveness, transduced by the Aδ fibers. As expected in view of the behavioral data, the addition of naloxone (2.75 mM) completely prevented the analgesic action of IRL-1620. Thus, these electrophysiological results confirm the behavioral observations that ET_{B} receptor activation inhibits ET-1-mediated nociception by a mechanism involving opioid receptors.

### Opioid Receptor Subtypes Involved in ET_{B}-mediated Analgesia

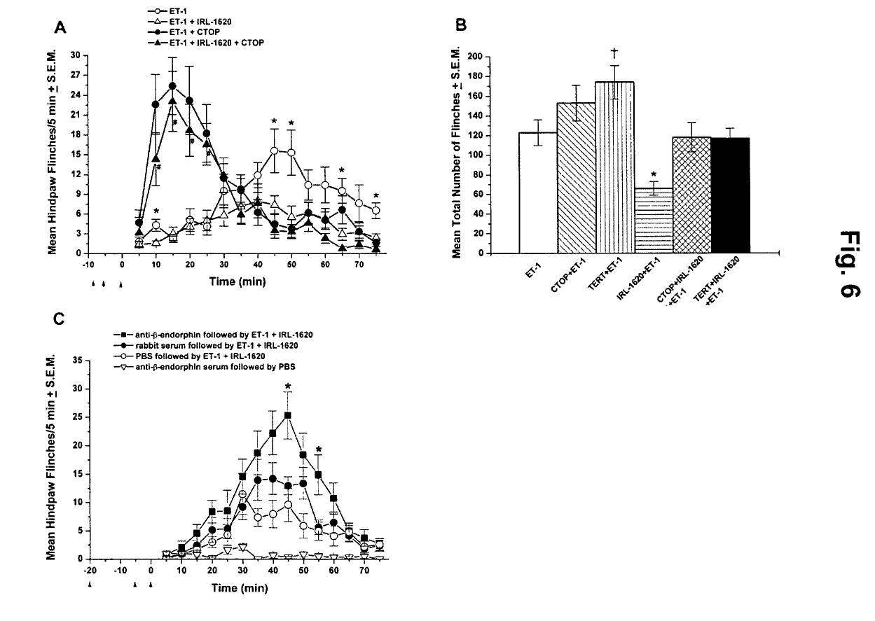

Selective opioid receptor antagonists were used to determine the specific opioid receptor subtype(s) responsible for modulating ET_{B}-mediated analgesia. The selective μ-opioid receptor antagonist, CTOP (18.8 nmoles), when co-injected with ET1 and IRL-1620, blocked the analgesic effects of IRL-1620. CTOP administration (FIGS. 6A and 6B) accelerated the time to MFF and resulted in a greater amount of flinching, measured both by the MFF and the total number of flinches over the entire test period, compared to the injection of ET-1 and IRL-1620 alone (31±4 flinches/5 minutes versus 16±2 flinches/5 minutes). The selective κ-opioid receptor antagonist, nor-BNI (3.4 mM), partially enhanced the flinching caused by ET-1 administration alone. Whereas, the δ-opioid receptor antagonist, naltrindole, did not affect flinching behavior following administration of either ET1 alone or the ET-1/IRL-1620 combination.

### Extracellular β-endorphin Mediates ET_{B} Receptor-mediated Analgesia

β-endorphin is a known product of cutaneous cells. Treatment of the plantar hindpaw by injection of antisera against β-endorphin (200 μg/hindpaw) prior to IRL-1620 and ET-1 administration completely abolished the analgesic effect of IRL-1620 at the time of MFF (FIG. 6C). Pretreatment with and equal volume of saline or normal rabbit serum had no effect on the onset or magnitude of MFF, and injection of the anti-β-endorphin serum without ET-1 did not cause any significant flinching behavior. These results demonstrate that the analgesic effect of ET_{B} receptor activation is transduced by the endogenous opioid agonist, β-endorphin.

### ET_{B} Receptor Activation Causes β-endorphin Release from Keratinocytes

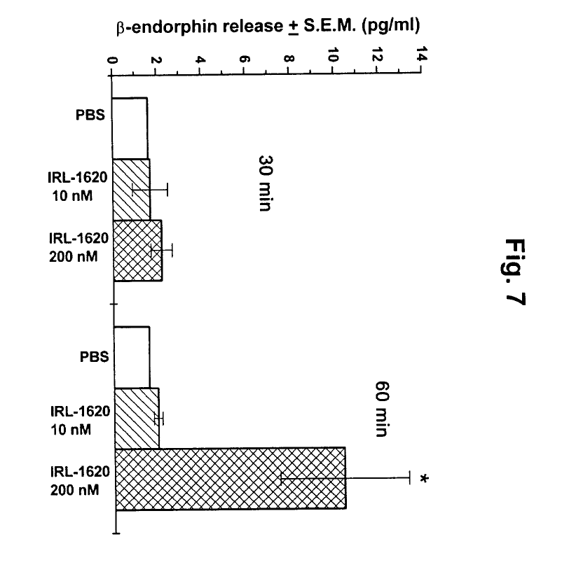

Cultured adult human primary keratinocytes, obtained during facelift procedures, were used to confirm that keratinocytes are the source of the extracellular β-endorphin that mediates ET_{B} receptor-induced analgesia. Cultured keratinocytes were transferred to PBS and incubated in the presence of either 10 or 200 nM IRL-1620 for thirty or sixty minutes. Subsequently, the β-endorphin concentration in the PBS was measured by radioimmunoassay. In the absence of IRL-1620, the basal level of β-endorphin was 1.76±0.08 pg/ml. Although 60 minute incubation with 10 nM IRL-1620 did not result in a significant change, 200 nM IRL-1620 caused a 5-fold increase in extracellular β-endorphin (10.5±3 pg/ml, p<0.02; FIG. 7). The intracellular concentration of β-endorphin in similarly treated cultured keratinocytes was 108±48 pg β-endorphin per 10⁵ cells.

These experiments demonstrate that human keratinocytes synthesize and store β-endorphin, which can be secreted upon ET_{B} receptor activation. Further, neighboring sensory neurons are known to contain μ-opioid receptors which, upon activation, mediate analgesia. We have also proven that this signaling pathway occurs in vivo, using a rat plantar hindpaw model of ET-1-induced pain.

### GIRK Channels in ET_{B} Receptor-mediated Analgesia

Endogenous opioids, such as β-endorphin, act on sensory neurons by modulating both calcium and potassium channels, thereby altering membrane excitability. Having discovered that β-endorphin and μ-opioid receptors are involved in ET_{B}-induced analgesia, we investigated the effects of ET1 signaling on G-protein coupled inwardly rectifying K⁺ (GIRK) channels. GIRK channels regulate membrane excitability and have been linked to μ-opioid receptor actions in heterologous expression systems and in rodents.

Prior and co-administration of tertiapin (20 pmol), a GIRK channel antagonist, with 2 nmol ET-1 significantly accelerated the development of hindpaw flinching. The time to reach MFF was shorter in the presence of tertiapin (41±4 versus 52±3 minutes; p<0.05). Tertiapin also caused a significant increase in the MFF (38±5 versus 24±2 flinches per 5 min; p<0.02; FIG. 4) and the total number of flinches (174±19 versus 123±13; p<0.05; FIG. 3) compared to administration of ET-1 alone. Tertiapin alone did not induce flinching behavior that was different from naloxone or PBS control.

Combination of Tertiapin and IRL-1620

Prior and co-administration of tertiapin (20 pmol) with IRL-1620 (2 nmol) and ET-1 (2 nmol) resulted in a complete reversal of the analgesic effects attributed to IRL-1620. Tertiapin administration in combination with ET1/IRL-1620, reversed the IRL-1620-induced reduction in the total number of flinches (117±10 versus 67±9 flinches; FIG. 3). The addition of tertiapin to the IRL-1620/ET-1 combination also reversed the reduction in MFF attributable to IRL-1620 (30±5 versus 16±2 flinches per 5 minutes; FIG. 4). These data indicate that a GIRK pathway mediates ET_{B} receptor-induced analgesia because the effects of ET_{B} receptor agonists can be completely blocked by a GIRK antagonist. In addition, these results indicate that the analgesic effects of ET_{B} receptor agonists may be augmented by co-administration of a compound that enhances potassium efflux across GIRK channels.

### ET-1 Pain Behavior in GIRK2 Knockout Mice

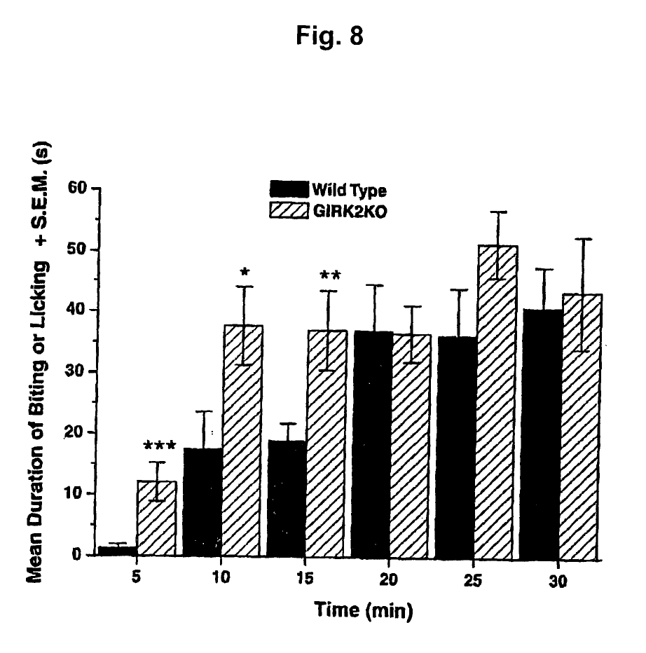

ET1-induced pain behavior was significantly increased in GIRK2 knockout mice, compared to wild-type control mice. The duration of biting and licking events was used as a measure of pain responsiveness following subcutaneous administration of ET1 (100 pmol) into the mouse plantar hindpaw. The duration of these events was assessed in five minute periods for 30 minutes after-ET-1 injection. The GIRK2 knockout mice had a faster onset and a greater total duration of biting and licking behavior (218±18 seconds versus 152±18 seconds; p<0.01; FIG. 8). These results further demonstrate that a GIRK receptor-dependent pathway modulates ET-1-mediated nociception.

### Protein Kinase C in ET-1-induced Nociception

Chelerythrine chloride (CL) is a specific and highly cell-permeant PKC inhibitor. Three different doses of CL (0.1, 0.5, and 0.8 μg) were injected subcutaneously into the rat plantar hindpaw two minutes prior to ET-1 (2 nmol). The 0.5 μg dose of CL produced a significant increase in ET1 evoked flinching behavior compared to control. The MFF was increase from 27±2 to 46±3 flinches per five minutes (p<0.0001). Likewise, 0.5 μg CL caused an increase in the total number of flinches over the 75 minute observation period (253±7 for CL versus 153±11 for control; p<0.0001). Administration of 0.1 μg CL was without effect, whereas 0.8 μg CL showed signs of toxicity.

Chelerythrine chloride also reversed the analgesic effect of ET_{B} receptor agonists. Significant increases were measured in both the MFF and total number of flinches when 0.5 μg CL was administered prior to a co-administration of ET-1 (2 nmol) and IRL-1620 (2 nmol) compared to pre-administration of and equal volume of water (MFF: 39±3 versus 22±3 flinches per five minutes (p<0.0002); total flinches: 207±10 versus 95±35 (p<0.0001)). Accordingly, it is expected that PKC enhancers will promote ET_{B} receptor-mediated analgesia.

### Other ET_{B} Receptor Agonists

While the present invention has been illustrated using the ET_{B} receptor agonist, IRL-1620, any other ET_{B} receptor agonist can be substituted for this compound and used in the methods and compositions of the invention. Examples of other useful ET_{B} receptor agonists are identified in Table 2. In addition, any ET_{A} receptor antagonist may be used in the invention; exemplary ET_{A} receptor antagonists are also identified in Table 2. These examples are not intended to be limiting.

TABLE 2
Endothelin Receptor Ligands

| | | |
|---|---|---|
| Endothelin-1 | SEQ ID NO:1 | Cys-Ser-Cys-Ser-Ser- |
| (ET-1) | | Leu-MCt-ASP-LYS-Glu- |
| | | Cys-Val-Tyr-Phe-Cys- |
| | | His-Leu-Asp-Ile-Ile- |
| | | Trp |

Endothelin-B Receptor Agonists

| | | |
|---|---|---|
| IRL-1620 | SEQ ID NO:2 | Suc-Asp-Glu-Glu-Ala- |
| | | Val-Tyr-Phe-Ala-His- |
| | | Leu-Asp-Ile-Ile-Trp |
| BQ-3020 | SEQ ID NO:3 | Ac-Leu-Met-Asp-Lys- |
| | | Glu-Ala-Val-Tyr-Phe- |
| | | Ala-His-Leu-Asp-Ile- |
| | | Ile-Trp |
| Sarafotoxin S6a | SEQ ID NO:4 | Cys-Ser-Cys-Lys-Asp- |
| | | Met-Thr-Asp-Lys-Glu- |
| | | Cys-Leu-Asn-Phe-Cys- |
| | | His-Gln-Asp-Val-Ile- |
| | | Trp |
| Sarafotoxin S6b | SEQ ID NO:5 | Cys-Ser-Cys-Lys-Asp- |
| | | Met-Thr-Asp-Lys-Glu- |
| | | Cys-Leu-Tyr-Phe-Cys- |
| | | His-Gln-Asp-Val-Ile- |
| | | Trp |
| Sarafotoxin S6c | SEQ ID NO:6 | Cys-Thr-Cys-Asn-Asp- |
| | | Met-Thr-Asp-Glu-Glu- |
| | | Cys-Leu-Asn-Phe-Cys- |
| | | His-Gln-Asp-Val-Ile- |
| | | Trp |
| Sarafotoxin S6d | SEQ ID NO:7 | Cys-Thr-Cys-Asn-Asp- |
| | | Met-Thr-Asp-Lys-Glu- |
| | | Cys-Leu-Tyr-Phe-Cys- |
| | | His-Glu-Asp-Ile-Ile- |
| | | Trp |

Endothelin-A Receptor Antagonists

| | | |
|---|---|---|
| altrasentan | | 2R-(4-methoxyphenyl)- |
| | | 4S-(1,3-benzodioxol- |
| | | 5-yl)-1- |
| (ABT-627) | | (N,N-di(n-butyl) |
| | | aminocarbonyl- |
| | | methyl)-pyrrolodine- |
| | | 3R-carboxylic acid |
| BQ-123 | SEQ ID NO:8 | Cyclo(D-Asp-Pro-D- |
| | | Val-Leu-D-Trp) |
| BQ-610 | | Homopiperidinyl- |
| | | carbonyl-Leu-D- |
| | | Trp(CHO)-D-Trp |
| FR139317 | | (Hexahydro-1H- |
| | | azepinyl)carbonyl- |
| | | Leu-D-(1-Me)- |
| | | Trp-D-3(2-pyridyl)- |
| | | Ala |
| PD-151242 | | (hexahydro-1H- |
| | | azepinyl)carbonyl- |
| | | Leu(1Me)-D-Trp-D-Tyr |
| TTA-386 | SEQ ID NO:9 | Hexamethyleniminocar- |
| | | bonyl-Leu-D-Trp-D- |
| | | Ala-beta-Ala-Tyr-D- |
| | | Phe |
| JKC-301 | SEQ ID NO:10 | Cyclo(D-Asp-Pro-D- |
| | | Ile-Leu-D-Trp) |
| JKC-302 | SEQ ID NO:11 | Cyclo(D-Ser-Pro-D- |
| | | Val-Leu-D-Trp) |
| BE-18257A | SEQ ID NO:12 | Cyclo(D-Glu-Ala-D- |
| | | Val-Leu-D-Trp) |
| BE-18257B | SEQ ID NO:13 | Cyclo(D-Glu-Ala-allo- |
| | | D-Ile-Leu-D-Trp) |
| BQ-485 | | Hexahydro-1H- |
| | | azepinyl-carbonyl- |
| | | Leu-D-Trp-D-Trp |

G-protein Coupled Inwardly Rectifying K⁺
Channel (GIRK) Antagonist

| | | |
|---|---|---|
| tertiapin | SEQ ID NO:14 | Ala-Leu-Cys-Asn-Cys- |
| | | Asn-Arg-Ile-Ile-Pro- |
| | | His-Met-Cys-Trp-Lys- |
| | | Lys-Cys-Gly-Lys-Lys |

### Dosages

The dosage of individual components or therapeutic combinations of the present invention can be readily determined by those skilled in the art of pain management. For example, the dose of an opioid receptor agonist administered according to the present invention will be the same or less than that which is practiced in the art.

### Formulation of Pharmaceutical Compositions

The administration of any compound of this invention may be by any suitable means that results in a concentration of the compound that is effective for the treatment of pain. The compound(s) may be contained in any appropriate amount in any suitable carrier substance, and is generally present in an amount of 1-95% by weight of the total weight of the composition. The composition may be provided in a dosage form that is suitable for the oral, parenteral (e.g., intravenous, intramuscular, or subcutaneous injection), rectal, or transdermal (topical) administration route. Thus, the composition(s) may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, or implants. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound (drug) substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain drug action during a predetermined time period by maintaining a relatively, constant, effective drug level in the body with concomitant minimization of undesirable side effects associated with fluctuations in the plasma level of the active drug substance (sawtooth kinetic pattern); (iv) formulations that localize drug action by, e.g., spatial placement of a controlled release composition adjacent to or in the diseased tissue or organ; and (v) formulations that target drug action by using carriers or chemical derivatives to deliver the drug to a particular target cell type.

Administration of compounds in the form of a controlled release formulation is especially preferred in cases in which the compound, either alone or in combination, has (i) a narrow therapeutic index (i.e., the difference between the plasma concentration leading to harmful side effects or toxic reactions and the plasma concentration leading to a therapeutic effect is small; in general, the therapeutic index, TI, is defined as the ratio of median lethal dose (LD50) to median effective dose (ED50)); (ii) a narrow absorption window in the gastro-intestinal tract; or (iii) a very short biological half-life so that frequent dosing during a day is required in order to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes.

### Solid Dosage Forms For Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug substance in a predetermined pattern (e.g., in order to achieve a controlled release formulation) or it may be adapted not to release the active drug substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active drug substance). The coating may be applied on the solid dosage form in a similar manner as that described in Encyclopedia of Pharmaceutical Technology, supra.

If more than one drug is administered simultaneously, the drugs may be mixed together in the tablet, or may be partitioned. In one example, a first drug is contained on the inside of the tablet, and a second drug is on the outside, such that a substantial portion of the second drug is released prior to the release of the first drug.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active drug by controlling the dissolution and/or the diffusion of the active drug substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Liquids for Oral Administration

Powders, dispersible powders, or granules suitable for preparation of an aqueous suspension by addition of water are convenient dosage forms for oral administration. Formulation as a suspension provides the active ingredient in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

### Parenteral Compositions

The compound(s) may also be administered parenterally by injection, infusion, or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active drug(s), the composition may include suitable parenterally acceptable carriers and/or excipients. The active drug(s) may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the suitable active drug(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug(s) may be incorporated in biocompatible carriers, liposomes, nanoparticles, implants, or infusion devices. Materials for use in the preparation of microspheres and/or microcapsules are, e.g., biodegradable/bioerodible polymers such as polygalactin, poly-(isobutyl cyanoacrylate), poly(2-hydroxyethyl-L-glutamine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)).

### Rectal Compositions

For rectal application, suitable dosage forms for a composition include suppositories (emulsion or suspension type), and rectal gelatin capsules (solutions or suspensions). In a typical suppository formulation, the active drug(s) are combined with an appropriate pharmaceutically acceptable suppository base such as cocoa butter, esterified fatty acids, glycerinated gelatin, and various water-soluble or dispersible bases like polyethylene glycols and polyoxyethylene sorbitan fatty acid esters. Various additives, enhancers, or surfactants may be incorporated.

### Percutaneous and Topical Compositions

The pharmaceutical compositions may also be administered topically on the skin for percutaneous (transdermal) absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

Examples of emulsifying agents are naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE™. Examples of chelating agents are sodium EDTA, citric acid, and phosphoric acid. Examples of gel forming agents are CARBOPOL™, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN™)).

The pharmaceutical compositions described above may be applied by means of special drug delivery devices such as dressings or alternatively plasters, pads, sponges, strips, or other forms of suitable flexible material.

### Controlled Release Percutaneous and Topical Compositions

There are several approaches for providing rate control over the release and transdermal permeation of a drug, including: membrane-moderated systems, adhesive diffusion-controlled systems, matrix dispersion-type systems, and microreservoir systems. A controlled release percutaneous and/or topical composition may be obtained by using a suitable mixture of the above-mentioned approaches.

In a membrane-moderated system, the active drug is present in a reservoir which is totally encapsulated in a shallow compartment molded from a drug-impermeable laminate, such as a metallic plastic laminate, and a rate-controlling polymeric membrane such as a microporous or a non-porous polymeric membrane (e.g., ethylene-vinyl acetate copolymer). The active compound is only released through the rate-controlling polymeric membrane. In the drug reservoir, the active drug substance may either be dispersed in a solid polymer matrix or suspended in a viscous liquid medium such as silicone fluid. On the external surface of the polymeric membrane, a thin layer of an adhesive polymer is applied to achieve an intimate contact of the transdermal system with the skin surface. The adhesive polymer is preferably a hypoallergenic polymer that is compatible with the active drug.

In an adhesive diffusion-controlled system, a reservoir of the active drug is formed by directly dispersing the active drug in an adhesive polymer and then spreading the adhesive containing the active drug onto a flat sheet of substantially drug-impermeable metallic plastic backing to form a thin drug reservoir layer. A matrix dispersion-type system is characterized in that a reservoir of the active drug substance is formed by substantially homogeneously dispersing the active drug substance in a hydrophilic or lipophilic polymer matrix and then molding the drug-containing polymer into a disc with a substantially well-defined surface area and thickness. The adhesive polymer is spread along the circumference to form a strip of adhesive around the disc.

In a microreservoir system, the reservoir of the active substance is formed by first suspending the drug solids in an aqueous solution of water-soluble polymer, and then dispersing the drug suspension in a lipophilic polymer to form a plurality of microscopic spheres of drug reservoirs.

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Other embodiments are within the claims.

What is claimed is:

1. A method for treating pain in a mammal, said method comprising administering to said mammal an analgesia-inducing amount of an endothelin-B receptor agonist.

2. The method of claim 1, wherein said mammal is a human.

3. The method of claim 1, wherein said pain is acute pain.

4. The method of claim 1, wherein said pain is caused by traumatic injury or surgery.

5. The method of claim 1, wherein said mammal is diagnosed as having psoriasis, scleroderma, or pruritis.

6. The method of claim 1, wherein said mammal has a thermal, chemical, or radiation burn of the cutaneous tissue.

7. The method of claim 6, wherein said mammal has a sunburn.

8. The method of claim 1, wherein said mammal is diagnosed as having cancer.

9. The method of claim 8, wherein said cancer is metastatic prostate or breast cancer.

10. The method of claim 1, wherein said mammal is diagnosed as having cardiovascular disease.

11. The method of claim 10, wherein said cardiovascular disease is myocardial infarction, angina, ischemic cardiovascular disease, peripheral vascular occlusive disease, or peripheral arterial occlusive disease.

12. The method of claim 1, wherein said mammal is diagnosed as having sickle cell anemia, migraine headache, inflammatory conditions of the skin or joints, or diabetic neuropathy.

13. The method of claim 1, wherein said endothelin-B receptor agonist is administered orally or by intravenous, intramuscular, or subcutaneous injection.

14. The method of claim 1, wherein said endothelin-B receptor agonist is administered topically.

15. The method of claim 1, wherein said endothelin-B receptor agonist is IRL-1620.

16. The method of claim 1, wherein said method further comprises administering to said mammal a second analgesia-inducing compound.

17. The method of claim 16, wherein said second analgesia-inducing compound is an endothelin-A receptor antagonist.

18. The method of claim 17, wherein said endothelin-A receptor antagonist is sulfisoxazole.

19. The method of claim 17, wherein said endothelin-A receptor antagonist is ABT-627.

20. The method of claim 16, wherein said second analgesia-inducing compound is an opioid receptor agonist.

21. The method of claim 20, wherein said opioid receptor agonist is morphine, codeine, hydrocodone, or oxycodone.

22. The method of claim 16, wherein said second analgesia-inducing compound is a GIRK channel activator.

23. The method of claim 16, wherein said second analgesia-inducing compound is a protein kinase C activator.

24. The method of claim 16, wherein said endothelin-B receptor agonist and said second analgesia-inducing compound are administered within one hour of each other.

25. The method of claim 24, wherein said endothelin-B receptor agonist and said second analgesia-inducing compound are administered simultaneously.

26. The method of claim 25, wherein said endothelin-B receptor agonist and said second analgesia-inducing compound are administered in the same pharmaceutical formulation.

27. A pharmaceutically acceptable composition comprising an endothelin-B receptor agonist.

28. The pharmaceutically acceptable composition of claim 27, wherein said composition is suitable for topical or oral administration or intravenous, intramuscular, or subcutaneous injection.

29. The pharmaceutically acceptable composition of claim 27, wherein said composition is suitable for topical administration.

30. The pharmaceutically acceptable composition of claim 27, wherein said composition is suitable for subcutaneous injection.

31. The pharmaceutically acceptable composition of claim 27, wherein said endothelin-B receptor agonist is IRL-1620.

32. The pharmaceutically acceptable composition of claim 27, wherein said composition further comprises a second analgesia-inducing compound.

33. The pharmaceutically acceptable composition of claim 32, wherein said second analgesia-inducing compound is an endothelin-A receptor antagonist.

34. The pharmaceutically acceptable composition of claim 33, wherein said endothelin-A receptor antagonist is sulfisoxazole.

35. The pharmaceutically acceptable composition of claim 33, wherein said endothelin-A receptor antagonist is ABT-627.

36. The pharmaceutically acceptable composition of claim 32, wherein said second analgesia-inducing compound is an opioid receptor agonist.

37. The pharmaceutically acceptable composition of claim 36, wherein said opioid receptor agonist is morphine, codeine, hydrocodone, or oxycodone.

38. The pharmaceutically acceptable composition of claim 32, wherein said second analgesia-inducing compound is a GIRK channel activator.

39. The pharmaceutically acceptable composition of claim 32, wherein said second analgesia-inducing compound is a protein kinase C activator.

40. The pharmaceutically acceptable composition of claim 29, wherein said composition is a wound dressing impregnated with said endothelin-B receptor agonist.

41. The pharmaceutically acceptable composition of claim 29, wherein said composition is a cream, lotion, gel, or ointment.
